(19) United States
(12) Patent Application Publication
(10) Pub. No.: US 2007/0184041 A1
(43) Pub. Date: Aug. 9, 2007

(54) METHODS AND COMPOSITIONS RELATED TO PRODUCTION OF COENZYME Q10

(76) Inventor: Adam M. Burja, Halifax (AU)

Correspondence Address: NEEDLE & ROSENBERG, P.C., SUITE 1000, 999 PEACHTREE STREET, ATLANTA, GA, 30309-3915, US

(21) Appl. No.: 11/671,281

(22) Filed: Feb. 5, 2007

Related U.S. Application Data
(60) Provisional application No. 60/771,962, filed on Feb. 9, 2006.

(51) Int. Cl. A61K 38/43; C12P 7/66; C12N 5/06

(52) U.S. Cl. 424/94.1; 435/133; 435/358

(57) ABSTRACT

Disclosed are methods, compounds, and compositions related to the production of coenzyme Q.

### I. CROSS-REFERENCE TO RELATED APPLICATIONS

This application claims priority to Provisional Application No. 60/771,962, filed Feb. 9, 2006, which application is hereby incorporated by this reference in its entirety.

### II. FIELD

The disclosed matter relates to compounds comprising coenzyme Q, including methods of making and using such compounds.

### III. BACKGROUND

Coenzymes Q occur in the majority of aerobic organisms, from bacteria to plants and animals. Two numbering systems exist for designating the number of isoprenoid units in the terpinoid “tail”: coenzyme Qₙ and coenzyme Q(x), where n refers to the number of isoprenoid side chains and x refers to the number of carbons in the terpinoid “tail” and can be any multiple of five. Thus, coenzyme Q₁₀ (also termed CoQ₁₀) refers to a coenzyme Q having 10 isoprenoid units in the “tail.” Since each isoprenoid unit has five carbons, CoQ₁₀ can also be designated coenzyme Q(50) or CoQ(50). The name CoQₙ can be used to generally refer to both the oxidized form and reduced form of the compound; alternatively, these specific forms can be individually designated CoQ_{nred} and CoQₙₒₓ. Chemically, CoQ₁₀ₒₓ, is known as 2,3-dimethyoxy-5-methyl-6-decaprenyl-1,4-benzoquinone, and its structural formula is:

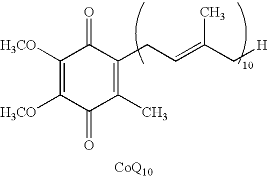

CoQ₁₀ is a model carrier of protons and electrons. It plays a vital role in the mitochondrial respiratory chain and oxidative phosphorylation. It was first isolated by researchers working at the Enzyme Institute of the University of Wisconsin (Crane, et al., BBA 25:220-1, 1975). Currently Japanese Kaneka Corp. supplies 60-70% of CoQ₁₀ sold in the USA.

The oxidized form of CoQ₁₀ (CoQ₁₀ₒₓ) has anti-atherogenic properties. Deficiencies in CoQ₁₀ₒₓ are associated with higher incidence of heart failure and other cardiovascular problems. Although CoQ₁₀ plays an important role in the development of cardiovascular disease, there have been data that suggest that the coenzyme also plays an important role in the nervous system. For example, CoQ₁₀ is believed to have beneficial effects in the prevention and treatment of Parkinson's disease, mitochondrial myopathies, and muscular dystrophy, etc.

Several attempts have been made to deliver benzendiol derivatives such as CoQ₁₀ to a subject. Selzer disclosed a liquid dietary CoQ₁₀ supplement based on vegetable oil-water emulsion. The absorption of CoQ₁₀ from this formulation was enhanced (U.S. Pat. No. 6,652,891 to Selzer et al.).

Natural Health Sciences together with General Nutrition Centers developed a blend of Pycnogenol, a French maritime pine bark extract, and CoQ₁₀ called PycnoQ10. Joint research executed at Showa Medical University, Tokyo, and State University of New York suggested that the combination protected 53% of blood lipids from oxidation compared to 30% when the ingredients were used separately. The product protected blood vessel integrity, blood lipid values, circulation, blood pressure, and platelet function. The activity is believed to be derived from the synergy of antioxidant properties.

In light of the numerous health benefits associated coenzyme Q compounds such as CoQ₁₀, what is needed in the art are new methods of preparing and using such compounds and compositions. The compounds, compositions, and methods disclosed herein meet these needs.

### IV. SUMMARY

In accordance with the purposes of the disclosed materials, compounds, compositions, articles, and methods, as embodied and broadly described herein, the disclosed subject matter, in one aspect, relates to compounds and compositions and methods for preparing and using such compounds and compositions.

Disclosed herein are methods of preparing an isoprenoid composition, the method comprising culturing ATCC 55366, and isolating the isoprenoid. Also disclosed are compounds prepared by this method. Also disclosed are nutritional supplements, delivery devices, and foodstuffs comprising the compounds.

Also disclosed are methods of lowering total cholesterol levels or triglyceride levels, increasing HDL levels, or a combination thereof in a subject, comprising the step of administering an effective amount of the compounds disclosed herein.

Also disclosed are methods of reducing hyperglycemia or hypercholesterolemia in a subject, comprising the step of administering an effective amount of the compounds disclosed herein.

Disclosed herein are methods for treating or preventing a mitochondrial condition or disease in a subject, comprising the step of administering to the subject an effective amount of the compounds disclosed herein.

Also disclosed are methods for increasing circulation or the immune system in a subject, comprising the step of administering to the subject an effective amount of the compounds disclosed herein.

Disclosed are methods for reducing the side effects of chemotherapy in a subject, comprising the step of administering to the subject an effective amount of the compounds disclosed herein.

Also disclosed are methods for treating or preventing degenerative heart disease in a subject, comprising the step of administering to the subject an effective amount of the compounds disclosed herein.

Also disclosed herein are pharmaceutical formulations comprising the compounds disclosed herein and a pharmaceutical carrier.

### V. DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, articles, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

A. General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an unsaturated fatty acid” includes mixtures of two or more such unsaturated fatty acids, reference to “the microcapsule” includes mixtures of two or more such microcapsules, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

The term “increase,” or other forms of increase, such as “increasing,” refers to an increase in an event or characteristic above basal levels, e.g., as compared to a control. The terms “reduces” or “lowers,” or other forms the words, such as “reducing,” “reduction,” or “lowering,” refers to a decrease in an event or characteristic below basal levels, e.g., as compared to a control.

By “control” is meant either a subject, organ, tissue, or cell lacking a disease or injury, or a subject, organ, tissue, or cell in the absence of a particular variable such as a therapeutic agent. A subject, organ, tissue, or cell in the absence of a therapeutic agent can be the same subject, organ, tissue, or cell before or after treatment with a therapeutic agent or can be a different subject, organ, tissue, or cell in the absence of the therapeutic agent. Comparison to a control can include a comparison to a known control level or value known in the art. Thus, basal levels are normal in vivo or in vitro levels prior to, or in the absence of, the addition of an agent (e.g., a therapeutic agent) or another molecule, or the absence of a particular state or characteristic for which the control is to be used. Those of skill in the art understand how to identify a control for a specific activity or situation.

By “prevent” or other forms of prevent, such as “preventing” or “prevention,” is meant to stop a particular event or characteristic, to stabilize or delay the development or progression of a particular event or characteristic, or to minimize the chances that a particular event or characteristic will occur. Prevention does not require comparison to a control as it is typically more absolute than, for example, reduce or lower. As used herein, something could be reduced or lowered but not prevented, but something that is reduced or lowered could also be prevented. Likewise, something could be prevented but not reduced or lowered, but something that is prevented could also be reduced or lowered. It is understood that where reduce, lowered, or prevent are used, unless specifically indicated otherwise, the use of the other two words is also expressly disclosed. Thus, if lowering cholesterol levels is disclosed, then reducing and preventing cholesterol levels are also disclosed, and the like.

By “treat” or other forms of treat, such as “treated” or “treatment,” is meant to administer a composition disclosed herein or to perform a method disclosed herein in order to reduce or prevent a particular characteristic or event (e.g., coronary artery disease).

Reference will now be made in detail to specific aspects of the disclosed materials, compounds, compositions, articles, and methods, examples of which are illustrated in the accompanying Examples.

B. Methods of Making

Prokaryotic microbes have been found to possess the ability to produce isoprenoid compounds. Specific examples of isoprenoid compounds include ubiquinone, including but not limited to ubiquinone-10 (CoQ₁₀). The disclosed bacteria can also produce antioxidants, such as, but not limited to, carotenoid compounds. Therefore, disclosed herein are methods for preparing compounds such as isoprenoids and carotenoids. Also, disclosed herein are compounds prepared by the methods disclosed herein.

The prokaryotic microbes that are capable of producing the compounds disclosed herein are found in the order Alphaproteobacteria, specifically Rhizobiales such as Methylobacteriaceae, and include the following species: Methylobacterium chloromehtanicum; Methylobacterium dichloromethanicum; Methylobacterium fugisawaense; Methylobacterium lusitanum; Methylobacterium mesophilicum; Methylobacterium nodulans; Methylobacterium organophilum; Methylobacterium podarium; Methylobacterium populum; Methylobacterium radiotolerans; Methylobacterium rhodesianum; Methylobacterium rhodinum; Methylobacterium specialis; Methylobacterium suomiense; Methylobacterium thiocyanatum; Methylobacterium zatmanii;and preferably Methylobacterium extorquens, including ATCC 55366.

Also disclosed are conditions for the isolation and growth of the bacteria. For example, disclosed are growth conditions for production of the disclosed isoprenoids and carotenoids, for example, both individually and cumulatively. It is understood that the microorganism and any clones, modified organisms or genes isolated from said microorganism set forth herein are also disclosed.

The bacteria can be grown without special conditions, and one of skill in the art would know how to culture such strains. Plates containing sources of carbon, nitrogen, inorganic salt, as well as substances necessary to stimulate growth can be used. As a source of carbon, any of the following can be used alone or in combination: L-mannose, L-fructose, galactose, glycerol, succinic acid, citric acid, acetic acid, or methanol. In one example, the bacteria are grown in CHOI medium. Optionally, the media can be supplemented with 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.5%, 3.0%, 4.0%, or 5.0% methanol, or any amount in between.

As a source of nitrogen, ammonium sulfate, ammonium phosphate, ammonium nitrate, peptone, and beef extract can be used. Inorganic salt, nitrate salt, magnesium salt, iron salt, and micromineral salts can also be used, for example. As a material to accelerate growth, amino acid, nucleic acid, vitamins, yeast extract, as well as wheat germ extract can be used.

In one example, the temperature can range from 20-40° C. For each strain of bacteria, a suitable temperature for growth and propagation can be selected. One of skill in the art can determine a suitable temperature. The pH of the culture can be from 6-8, and can vary to determine the best outcome for production. If ammonium salt is used as a source of nitrogen, as the bacteria multiply, the pH of the growth fluid drops, and so ammonium or potassium (for example) can be added to maintain a consistent pH.

Cultivation of the biomass containing the isoprenoid/carotenoid can be periodical or continuous. In one example, CoQ₁₀ can be extracted from the biomass present on the media periodically. Methods of extraction are well known in the art, and an example of such extraction can be found in Example 1. A variety of procedures can be employed in the recovery of the resultant cellular biomass from fermentation in various culture media, such as by filtration or centrifugation. The product can then be washed, frozen, lyophilized, or spray dried, and stored under a non-oxidizing atmosphere to eliminate the presence of oxygen, prior to incorporation into a processed food or feed product.

Separation and extraction of CoQ₁₀ can be achieved using standard methods known in the art.

Also disclosed are processes for the production of microbial biomass containing the compounds disclosed herein, along with a process for preparing these compounds utilizing the microorganisms.

In addition, disclosed are isoprenoids, including CoQ₁₀, and carotenoids produced by the disclosed microorganisms, as well as various feedstuffs, nutraceuticals, pharmaceutical and food supplemented with the lipids and antioxidants, as well as a process for utilizing these compounds as an additive for various feedstuffs and foods.

Also in certain aspects are methods where the methods of producing CoQ₁₀ as discussed herein are combined methods of producing other compositions, such as foodstuffs, nutriceuticals, pharmaceuticals, caps, soft caps, gel caps, soft gel caps, microcaps, and the such. The methods involve at least one further step of combining the CoQ₁₀ as produced herein with one or more of another substance as discussed herein. Methods for the combining the compositions are known. The produced CoQ₁₀ can be combined with any known substance such as fish oil, derivatives of fish oil, such as EPA, or DHA, as well as with for example, other anti-oxidants such as other isoprenoids. It is understood that the method steps can be achieved by, for example, incubating, mixing, adding, admixing, or other ways of forming the combined compositions.

C. Compositions

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and a number of modifications that can be made to a number of components or residues of the compound are discussed, each and every combination and permutation that are possible are specifically contemplated unless specifically indicated to the contrary. Thus, if a class of components or residues A, B, and C are disclosed as well as a class of components or residues D, E, and F, and an example of a combination compound A-D is disclosed, then even if each is not individually recited, each is individually and collectively contemplated. Thus, in this example, each of the combinations A-E, A-F, B-D, B-E, B—F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B—F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Certain materials, compounds, compositions, and components disclosed herein can be obtained commercially or can be readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

D. Supplements

Disclosed herein are nutritional supplements. A nutritional supplement is any compound or composition that can be administered to or taken by a subject to provide, supply, or increase a nutrient(s) (e.g., vitamin, mineral, essential trace element, amino acid, peptide, nucleic acid, oligonucleotide, lipid, cholesterol, steroid, carbohydrate, and the like). In one aspect, disclosed herein are nutritional supplements comprising any of the compounds disclosed herein. For example, a nutritional supplement can comprise any compound comprising CoQ₁₀.

The nutritional supplement can comprise any amount of the compounds disclosed herein, but will typically contain an amount determined to supply a subject with a desired dose of a CoQ₁₀. The exact amount of compound required in the nutritional supplement will vary from subject to subject, depending on the species, age, weight and general condition of the subject, the severity of the dietary deficiency being treated, the particular mode of administration, and the like. Thus, it is not possible to specify an exact amount for every nutritional supplement. However, an appropriate amount can be determined by one of ordinary skill in the art using only routine experimentation given the teachings herein. In one specific example, a nutritional supplement can comprise from about 0.05 to about 20%, from about 1 to about 7.5%, or from about 3 to about 5% by weight of the compound. In another example, the nutritional supplement can comprise from about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, or 20.0% by weight of the compound, where any of the stated values can form an upper or lower endpoint when appropriate. In another aspect, when the nutritional supplement, the supplement can be composed of up to 100% of the supplement.

The nutritional supplement can also comprise other nutrient(s) such as vitamins trace elements, minerals, and the like. Further, the nutritional supplement can comprise other components such as preservatives, antimicrobials, anti-oxidants, chelating agents, thickeners, flavorings, diluents, emulsifiers, dispersing aids, and/or binders.

The nutritional supplements are generally taken orally and can be in any form suitable for oral administration. For example, a nutritional supplement can typically be in a tablet, gel-cap, capsule, liquid, sachets, or syrup form.

E. Pharmaceutical formulation

Also disclosed herein are pharmaceutical formulations. In one aspect, a pharmaceutical formulation can comprise any of the compounds disclosed herein with a pharmaceutically acceptable carrier. For example, a pharmaceutical formulation can comprise a compound comprising CoQ₁₀ and a pharmaceutically acceptable carrier. The disclosed pharmaceutical formulations can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) Gennaro, ed., Mack Publishing Company, Easton, Pa., 1995, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, antiinflammatory agents, anesthetics, and the like.

The pharmaceutical formulation can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compounds can be administered orally, intravenously, intraperitoneally, intramuscularly, subcutaneously, intracavity, or transdermally.

Pharmaceutical formulations for oral administration include, but are not limited to, powders or granules, suspensions or solutions in water or non-aqueous media, capsules, sachets, or tablets. Thickeners, flavorings, diluents, emulsifiers, dispersing aids, anti-oxidants, or binders may be desirable.

Pharmaceutical formulations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

F. Delivery Devices

Any of the compounds described herein can be incorporated into a delivery device. Examples of delivery devices include, but are not limited to, microcapsules, microspheres, nanospheres or nanoparticles, liposomes, noisome, nanoerythrosome, solid-liquid nanoparticles, gels, gel capsules, tablets, lotions, creams, sprays, emulsions. Other examples of delivery devices that are suitable for non-oral administration include pulmospheres.

1. Targeted Delivery

The disclosed liposomes and microcapsules can be targeted to a particular cell type, such as islets cells, via antibodies, receptors, or receptor ligands. The following references are examples of the use of this technology to target specific tissue (Senter, et al., Bioconjugate Chem 2:447-51, 1991; Bagshawe, Br J Cancer 60:275-81, 1989; Bagshawe, et al., Br J Cancer 58:700-3, 1988; Senter, et al., Bioconjugate Chem 4:3-9, 1993; Battelli, et al., Cancer Immunol Immunother 35:421-5, 1992; Pietersz and McKenzie, Immunolog Reviews 129:57-80, 1992; and Roffler, et al., Biochem Pharmacol 42:2062-5, 1991). These techniques can be used for a variety of other specific cell types.

G. Foodstuffs

Also disclosed herein are foodstuffs comprising any of the compositions disclosed herein. By “foodstuff” is meant any article that can be consumed (e.g., eaten, drank, or ingested) by a subject. In one aspect, the microcapsules can be used as nutritional supplements to a foodstuff. For example, the compositions can be loaded with vitamins, and other compounds that provide health benefits. In one aspect, the foodstuff is a baked good, a pasta, a meat product, a frozen dairy product, a milk product, a cheese product, an egg product, a condiment, a soup mix, a snack food, a nut product, a plant protein product, a hard candy, a soft candy, a poultry product, a processed fruit juice, a granulated sugar (e.g., white or brown), a sauce, a gravy, a syrup, a nutritional bar, a beverage, a dry beverage powder, a jam or jelly, a fish product, or pet companion food. In another aspect, the foodstuff is bread, tortillas, cereal, sausage, chicken, ice cream, yogurt, milk, salad dressing, rice bran, fruit juice, a dry beverage powder, rolls, cookies, crackers, fruit pies, or cakes.

H. Methods of Use

Also disclosed herein, in one aspect, are methods of preventing a mitochondrial condition or disease in a subject by administering an effective amount of any of the compounds described herein to the subject. An example of a mitochondrial condition includes, but is not limited to, mitochondriopathy. Mitochondriopathy can be characterized by a CoQ₁₀ deficiency, ubiquinone-cytochrome c oxidoreductase deficiency, cytochrome c oxidase deficiency, chronic progressive external ophthalmoplegia syndrome, age-related macular degeneration, neuropathy, ataxia, or retinis Pigmentosa.

In another aspect, disclosed herein are methods of increasing circulation in a subject by administering an effective amount of any compound comprising any of the compounds described herein to the subject. In still another aspect, disclosed herein are methods of increasing the immune system in a subject by administering an effective amount of any compound comprising any of the compounds described herein to the subject. Principles and examples of use of immunostimulants and immunomodulators are described for instance in: “Immunostimulants Now and Tomorrow” (Azuma I, Jolles G, eds.), Japan Scientific Societies Press,Tokyo, 1987; Hadden J W (1992) “Classification of Immunotherapeutic Agents In Developments of Biological Standardization,” Vol 77, (eds Brown F, Revillard J P): 5-15; Karger, Basel; Galeotti M (1998) “Some Aspects of the Application of Immunostimulants and a Critical Review of Methods for their Evaluation” J Appl Ichtuol 189-199; Halperin S A, Smith B S, Nolan C, Shay J, Kralovec J (2003) “Randomized, Double-Blind, Placebo-Controlled Trial of the Safety and Immunostimmulatory Effect of a Chlorella-Derived Food Supplement in Healthy Adults Undergoing Influenza Immunization” Can Med Assoc J 169: 111-117), which are incorporated by reference in their entireties. In yet another aspect, disclosed herein are methods of reducing the side effects of chemotherapy in a subject by administering an effective amount of any compound comprising any of the compounds described herein to the subject. In still another aspect, disclosed herein are methods of treating or preventing degenerative heart disease in a subject by administering an effective amount of any compound comprising any of the compounds described herein to the subject.

Further, disclosed herein are methods of treating other conditions or diseases in a subject by administering an effective amount of CoQ₁₀ to the subject. Such other conditions or diseases include, but are not limited to, cystic fibrosis, asthma, periodontal (gum) disease, Alzheimer's disease, poor athletic performance, breast cancer, chronic obstructive pulmonary disease (COPD), HIV, male infertility, insulin resistance syndrome (Syndrome X), lung cancer, and prostate cancer.

The disclosed compounds herein can be used neat or in combination with some other component. For example, the compounds can be used in the disclosed methods in the form of any of the nutritional supplements disclosed herein. In another example, the compounds can be used in the disclosed methods in the form of any of the pharmaceutical formulations disclosed herein. In still another example, the compounds can be encapsulated in any of the microcapsules or liposomes disclosed herein, or incorporated into any foodstuff disclosed herein and used in the disclosed methods.

It is contemplated that the methods disclosed herein can be accomplished by administering various forms of the compounds disclosed herein. For example, one can administer any of the pharmaceutical formulations with any of the foodstuffs disclosed herein. In another example, one can administer any of the microcapsules with any of the nutritional supplements disclosed herein. In yet another example, one can administer any of the pharmaceutical formulations with any of the microcapsules and nutritional supplement disclosed herein, and the like.

1. Dosage

When used in the above described methods or other treatments, or in the nutritional supplements, pharmaceutical formulations, microcapsules, liposomes, or foodstuffs disclosed herein, an “effective amount” of one of the disclosed compounds can be employed in pure form or, where such forms exist, in pharmaceutically acceptable salt form and with or without a pharmaceutically acceptable excipient, carrier, or other additive.

The specific effective dose level for any particular subject will depend upon a variety of factors including the condition or disease being treated and the severity of the condition or disease; activity of the specific compound employed; the specific composition employed; the age, body weight, general health, sex and diet of the subject; the time of administration; the route of administration; the rate of excretion of the specific compound employed; the duration of the treatment; drugs used in combination or coincidential with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose.

The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products. A typical daily dosage of the compounds disclosed herein used alone might range from about 10 mg to up to 500 mg (CoQ₁₀ content only) or more per day, depending on the factors mentioned above.

2. Administration and Delivery

In one aspect, disclosed herein are uses of a microcapsule to deliver a loading substance to a subject, wherein the microcapsule contains any of the compounds disclosed herein. Also disclosed are methods for delivering a compound to a subject by administering to the subject any of the microcapsules disclosed herein. Further, disclosed are methods for delivering a compound disclosed herein to a subject by administering to the subject any of the nutritional supplements, pharmaceutical formulations, liposomes, and/or foodstuffs disclosed herein.

The compounds disclosed herein (including nutritional supplements, microcapsules, liposomes, and pharmaceutical formulations) can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

### VI. EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### 1. Example 1

### Co-Enzyme Q10 in Methylobacterium extorquens ATCC 55366

It was found that a facultative methylotrophic micro-organism, Methylobacterium extorquens ATCC 55366, when grown on CHOI medium supplemented with 1% methanol, produced biomass having a heavy pink pigmentation. Four samples were assayed (2×Q10 samples: 1.5 g & 2.7 g freeze-dried biomass; 2×carotenoid samples: ext41 T2 A & B from 50 ml cultures, approximately 0.5 g/L dry weight). These samples were processed for CoQ₁₀ and carotenoid analysis. Details are as follows:

| | | | |
|---|---|---|---|
| SS-2-57-1 | Q10 #1 (ext 741) 26/07/05 | 0.4026 g | 1 ml final volume |
| SS-2-57-2 | Duplicate of SS-2-57-1 | 0.3969 g | 1 ml final volume |
| SS-2-57-3 | Q10 #2 (ext 741) 26/07/05 | 0.3882 g | 1 ml final volume |
| SS-2-57-4 | Duplicate of SS-2-57-3 | 0.3995 g | 1 ml final volume |

Biomass samples were extracted and analyzed for co-enzyme Q₁₀ production. Additionally, standards (coenzyme Q₆, Q₉ and Q₁₀) were made up as 1, 2.5, 5, and 10 μg/mL solutions in Acetone with 0.5% BHA/BHT (1:1). Moreover, biomass extractions were carried out on all samples (SS-2-57-1 to 4) by adding 3 ml of MeOH:Hexane (3:2; v/v), vortexing for a minute, 8 ml of hexane is then added and again vortexed for a minute. Samples were then centrifuged for 10 minutes at 4,000 RPM, with hexane layer then transferred to scintillation vials. This process was then repeated, hexane extracts pooled and reduced to dryness under N₂ evaporation and resuspended in 0.25 acetone. Subsequently, 10 ml of each sample was analyzed for coenzyme content using an Agilent 1100 HPLC (Phenomenex bondclone C₁₈, 10 μm, 3.9×300 mm column, Waters sentry, symmetry C₁₈, 5 μm, 3.9×20 mm guard column; mobile phase: methanol:isopropanol (7:3; v/v); 0.6 ml/min flow rate, at 275 nm run for 35 minutes). Calibration curve was created using a 33.33 μg/ml CoQ6, 9 & 10 standard mix and an individual 10 μg/ml CoQ10 solution (both in acetone). After HPLC analysis using the calibration curve and co-injection with CoQ10 standard, the following concentrations of CoQ10 were found.

| Sample | per 1 ml | per gram | Average |
|---|---|---|---|
| SS-2-57-1 | 78.38 μg | 194.68 μg | 198.26 μg |
| SS-2-57-2 | 80.11 μg | 201.84 μg | |
| SS-2-57-3 | 79.75 μg | 205.44 μg | 215.98 μg |
| SS-2-57-4 | 90.49 μg | 226.51 μg | |

Results were then confirmed by outsourced analysis at KGK Synergize Inc. Freeze dried microbial biomass was sent for analysis using LC/MS/MS using Varian LC1200 in triplicate (detection limit: 0.1 ng). Standards to develop calibration curve used included CoQ6 (Oxidized), CoQ6 (Reduced), CoQ9 (Oxidized), CoQ9 (Reduced), CoQ10 (Oxidized) and CoQ10 (Reduced) at 1.0ng/μL, 0.1ng/μL and 0.01ng/μL concentrations.

| | |
|---|---|
| BRI-Q10-1 | BRI-Q10-2 |

| | Reduced | Total | Reduced | Total |
|---|---|---|---|---|
| Coenzyme | μg/g | μg/g | μg/g | μg/g |
| CoQ6 | ND | 10.9 | ND | ND |
| CoQ9 | ND | 20.3 | ND | 6.1 |
| CoQ10 | 46.3 | 269.2 | 49.5 | 276.9 |

What is claimed is:

1. A method of preparing an isoprenoid composition, the method comprising:
culturing ATCC 55366, and isolating the isoprenoid.

2. The method of claim 1, wherein the isoprenoid is a coenzyme.

3. The method of claim 1, wherein the isoprenoid is Coenzyme-Q₁₀ (CoQ₁₀).

4. The method of claim 1, wherein ATCC 55366 is cultured in CHOI medium.

5. A compound prepared by the method of claim 1.

6. A nutritional supplement comprising the compound of claim 5.

7. The nutritional supplement of claim 6, comprising from about 0.05% to 20% by weight of the compound.

8. The nutritional supplement of claim 7 comprising from about 1% to 7.5% by weight of the compound.

9. The nutritional supplement of claim 6, wherein the supplement comprises up comprises to or less than or equal to 100% by weight of the compound.

10. The nutritional supplement of claim 6, wherein the supplement is in the form of a tablet, gel-cap, capsule, liquid, or syrup.

11. A delivery device comprising the compound of claim 5.

12. A foodstuff comprising the compound of claim 5.

13. A method of lowering total cholesterol levels or triglyceride levels, increasing HDL levels, or a combination thereof in a subject, comprising the step of administering an effective amount of the compound of claim 5.

14. A method of reducing hyperglycemia in a subject, comprising the step of administering an effective amount of the compound of claim 5.

15. A method of reducing hypercholesterolemia in a subject, comprising the step of administering an effective amount of the compound of claim 5.

16. A method for treating or preventing a mitochondrial condition or disease in a subject, comprising the step of administering to the subject an effective amount of the compound of claim 5.

17. The method of claim 16, wherein the condition is a mitochondriopathy.

18. The method of claim 17, wherein the mitochondriopathy is Coenzyme Q₁₀ deficiency, ubiquinone-cytochrome c oxidoreductase deficiency, cytochrome c oxidase deficiency, chronic progressive external ophthalmoplegia syndrome, age-related macular degeneration, neuropathy, ataxia, or retinis Pigmentosa.

19. A method for increasing circulation in a subject, comprising the step of administering to the subject an effective amount of the compound of claim 5.

20. A method for increasing the immune system in a subject, comprising the step of administering to the subject an effective amount of the compound of claim 5.

21. A method for reducing the side effects of chemotherapy in a subject, comprising the step of administering to the subject an effective amount of the compound of claim 5.

22. A method for treating or preventing degenerative heart disease in a subject, comprising the step of administering to the subject an effective amount of the compound of claim 5.

23. A pharmaceutical formulation comprising the compound of claim 5 and a pharmaceutical carrier.

24. A method of producing a dietary supplement comprising combining fish oil and CoQ₁₀, wherein the CoQ₁₀ is produced using the method of claim 1.

25. The method of claim 24, wherein the dietary supplement comprises a soft gel cap.
